(19) United States
(12) Patent Application Publication
(10) Pub. No.: US 2026/0357335 A1
(43) Pub. Date: Nov. 12, 2026

(54) METHODS AND COMPOSITIONS FOR PROFILING THE ENDS OF A CELL-FREE DNA MOLECULE

(71) Applicant: Foundation Medicine, Inc., Boston, MA (US)

(72) Inventors: Yu ZHENG, Lexington, MA (US); Jun ZHOU, Belmont, MA (US); William L. CAMARA, Holliston, MA (US); Christine M. MALBOEUF, Sharon, MA (US)

(73) Assignee: Foundation Medicine, Inc., Boston, MA (US)

(21) Appl. No.: 19/139,220

(22) PCT Filed: Dec. 15, 2023
(86) PCT No.: PCT/US2023/084329

§ 371 Date: Jun. 13, 2025

Related U.S. Application Data
(60) Provisional application No. 63/433,681, filed on Dec. 19, 2022.

(51) Int. Cl. C12Q 1/6869; C12N 9/12; C12N 15/10

(52) U.S. Cl. CPC C12Q 1/6869; C12N 9/1205; C12N 15/10; C12Y 207/01078

(57) ABSTRACT

Nucleic acid sequencing constructs and methods of making nucleic acid sequencing constructs for determining nucleic acid duplex, such as cell-free DNA, topology, including a length or sequence of a 5′ or 3′ overhang are described herein. Also described are methods and systems for analyzing sequencing data obtained from such nucleic acid sequencing constructs to detect a 5′ or 3′ overhang length or sequence.

### CROSS-REFERENCE TO RELATED APPLICATIONS

This application claims the priority benefit of U.S. Provisional Patent Application No. 63/433,681, filed Dec. 19, 2022, the entire contents of which are incorporated herein by reference for all purposes.

### FIELD OF THE INVENTION

Disclosed herein are methods and systems for making a sequencing construct that includes a DNA duplex, such as cell-free DNA (cfDNA), and analyzing said DNA duplex to determine topological and/or duplexed sequence information. Certain aspects of the disclosure relate more specifically to methods and systems for making a sequencing construct for determining and/or analyzing the 5′ and 3′ overhangs from the DNA duplex (e.g., cfDNA).

### BACKGROUND

Obtaining sequence information from paired nucleic acid strands (e.g., a nucleic acid duplex, such as cell-free DNA) is often challenging because sequencing technologies generally rely on single stranded sequencing. The top and bottom strands of a DNA sequence, for example, are generally sequenced by disrupting the duplex and sequencing each strand independently. This duplex separation can result in loss of significant information about the duplex molecule, including molecular topology. Further, dissociation of the two strands can result in a loss of correspondence of sequence information between the two strands, which often relies on the use of complex molecular barcoding techniques (such as unique molecular identifiers) to retain.

Cell-free DNA (cfDNA) molecules are free-floating double stranded DNA molecules (dsDNA or duplex DNA) found in the blood stream, typically as the result of cell apoptosis or necrosis, particularly in the context of disease. These degraded linear DNA fragments are often approximately 50-300 base pairs in length. Most commonly, cfDNA is assayed for cancer screening at early stages in disease progression by analyzing the cfDNA sequences to identify cancer-associated mutations. Native cfDNA often have DNA ends where either the 5′ or 3′ end overhangs the complimentary strand, thereby resulting in jagged ends. Standard DNA sequencing methods generally include sequencing DNA with blunt ends, which involve the removal of these native topologies during the end repair step of traditional double stranded library construction. As a result, all information about any jagged end lengths and sequences, native end sequences, gaps, and nicks that were present in the cfDNA and the opportunity to identify any possible relevance of this information for cancer diagnosis and cancer care are lost.

### SUMMARY OF THE INVENTION

Described herein is a method of making a nucleic acid construct comprising: directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule, to provide a circular nucleic acid construct; and converting (i) non-methylated cytosine residues in the circular nucleic acid construct into uracil residues, or (ii) methylated cytosine residues in the circular nucleic acid construct into uracil residues or dihydrouracil residues, wherein a duplex structure of the DNA duplex molecule is transformed into a single-stranded structure. In some implementations, the DNA duplex molecule is a cell-free DNA duplex molecule. In some implementations, the method comprises phosphorylating the 5′ end of the first strand of the DNA duplex molecule and the 5′ end of the second strand of DNA duplex molecule. In some implementations, the 5′ end of the first strand of the DNA duplex molecule and the 5′ end of the second strand of the DNA duplex molecule are phosphorylated using a T4 polynucleotide kinase. In some implementations, the 3′ end of the first strand of the DNA duplex molecule is directly attached to the 5′ end of the second strand of the DNA duplex molecule, and the 3′ end of the second strand of the DNA duplex molecule is directly attached to the 5′ end of the first strand of the DNA duplex molecule, using a single-stranded DNA (ssDNA) ligase.

In some implementations, the method comprises fraying the ends of the DNA duplex molecule while maintaining a partial duplex of the DNA duplex molecule prior to directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule. In some implementations, the fraying comprises heating the DNA duplex molecule or chemically denaturing the DNA duplex molecule.

In some implementations of the method, non-methylated cytosine residues in the circular nucleic acid construct are converted into uracil residues using a bisulfite reaction. In some implementations of the method, non-methylated cytosine residues in the circular nucleic acid construct are converted into uracil residues using an enzymatic reaction. In some implementations of the method, methylated cytosine residues in the circular nucleic acid construct are converted into dihydrouracil residues or uracil residues using TET-assisted bisulfite treatment or oxidative bisulfite treatment.

In some implementations, the method comprises amplifying the circular converted nucleic acid construct, thereby forming a linear converted nucleic acid construct, wherein the uracil residues or dihydrouracil residues in the circular converted nucleic acid construct are recognized as thymine residues in the amplifying. In some implementations of the method, the amplifying targets a subgenomic interval within a genome. In some implementations of the method, the linear converted nucleic acid construct is a rolling circle amplification (RCA) product. In some implementations of the method, the linear converted nucleic acid construct is a double-stranded polymerase chain reaction (PCR) product. In some implementations of the method, the linear converted nucleic acid construct is a double-stranded inverse PCR product. In some implementations of the method, the amplifying comprises using a first primer targeting a converted first sequence and a second primer targeting a converted second sequence.

Also described herein is a nucleic acid construct made according to any of the above methods.

Further described herein is a method comprising sequencing the linear converted nucleic acid construct made according to any of the above methods to provide sequencing data. In some implementations of the method, the sequencing data comprises paired-end sequencing data comprising a first and a second read. In some implementations, the method further comprises aligning a first portion of the first read and a first portion of the second read to a first converted nucleic acid reference sequence, and aligning a second portion of the first read and a second portion of the second read to second converted nucleic acid reference sequence, wherein the first converted nucleic acid reference sequence and the second converted nucleic acid reference sequence are complements to each other when compared in an unconverted state. In some implementations, the method further comprises determining a set of genomic coordinates within the first and second converted nucleic acid reference sequences, comprising a first genomic coordinate for the first portion of the first read, a second genomic coordinate for the first portion of the second read, a third genomic coordinate for the second portion of the first read, and a fourth genomic coordinate for the second portion of the second read. In some implementations, the method further comprises determining, based on the set of genomic coordinates, a presence or absence of a 3′ overhang for the first strand of the DNA duplex, a 5′ overhang for the first strand of the DNA duplex, a 3′ overhang for the second strand of the DNA duplex, and/or a 5′ overhang for the second strand of the DNA duplex.

In some implementations, the method further comprises determining, based on the set of coordinates, a 3′ overhang length for the first strand of the DNA duplex molecule, a 5′ overhang length for the first strand of the DNA duplex molecule, a 3′ overhang length for the second strand of the DNA duplex molecule, and/or a 5′ overhang length for the second strand of the DNA duplex molecule.

In some implementations, the method further comprises determining a methylation status for one or more bases in the DNA duplex molecule.

In some implementations, the method further comprises determining a methylation status for one or more bases in a 3′ overhang or a 5′ overhang of the DNA duplex molecule.

In some implementations, the DNA duplex molecule is obtained from an individual, and the method further comprises comprising generating a DNA duplex molecule overhang profile for the individual comprising information about overhangs for a plurality of DNA duplex molecules. In some implementations, the information comprises, for a plurality of DNA duplex molecules, the 3′ overhang length for the first strand of the DNA duplex molecule, a 5′ overhang length for the first strand of DNA duplex molecule, a 3′ overhang length for the second strand of the DNA duplex molecule, and/or a 5′ overhang length for the second strand of the DNA duplex molecule. In some implementations, the information comprises a sequence of a 5′ overhang or a 3′ overhang. In some implementations, the information comprises (1) a ratio, or a distribution of ratios, of the a 5′ overhang length for the first strand of DNA duplex molecule to a 3′ overhang length for the first strand of DNA duplex molecule, (2) a ratio, or a distribution of ratios, of the a 5′ overhang length for the first strand of DNA duplex molecule to a 3′ overhang length for the second strand of DNA duplex molecule, (3) a ratio, or a distribution of ratios, of the a 5′ overhang length for the first strand of DNA duplex molecule to a 5′ overhang length for the second strand of DNA duplex molecule, (4) a ratio, or a distribution of ratios, of the a 3′ overhang length for the first strand of DNA duplex molecule to a 5′ overhang length for the second strand of DNA duplex molecule, or (5) a ratio, or a distribution or ratios, of the a 3′ overhang length for the first strand of DNA duplex molecule to a 3′ overhang length for the second strand of DNA duplex molecule. In some implementations, the information comprises a ratio of (1) a 5′ overhang length for the first strand of DNA duplex molecule, a 3′ overhang length for the first strand of DNA duplex molecule, a 5′ overhang length for the second strand of DNA duplex molecule, a 3′ overhang length for the second strand of DNA duplex molecule to (2) a duplex length.

In some implementations, the method further comprises comparing the DNA duplex molecule overhang profile to a reference DNA duplex molecule overhang profile. In some implementations, the reference DNA duplex molecule overhang profile is based on DNA duplex molecules from a normal sample or a plurality of normal samples. In some implementations, the reference DNA duplex molecule overhang profile is based on DNA duplex molecules from a normal sample, wherein the normal sample is synthetically created from a plurality of individuals. In some implementations, the reference DNA duplex molecule overhang profile is based on DNA duplex molecules from a sample obtained from an individual with cancer or a plurality of individuals with cancer. In some implementations, the reference DNA duplex molecule overhang profile is based on DNA duplex molecules from a sample obtained from an individual with an abnormal fetus or a plurality of individuals with an abnormal fetus. In some implementations, the reference DNA duplex molecule overhang profile is based on DNA duplex molecules from a sample obtained from an individual that received a stable transplant or a plurality of individuals that received a stable transplant. In some implementations, the reference DNA duplex molecule overhang profile is based on DNA duplex molecules from a match normal sample obtained from the individual. In some implementations, the reference DNA duplex molecule overhang profile is based on DNA duplex molecules from a prior sample obtained from the individual.

In some implementations of any of the above methods, the DNA duplex molecule is obtained from a blood, plasma, serum, saliva, pleural fluid, or cerebrospinal fluid sample.

In some implementations of any of the above methods, the DNA duplex molecule is obtained from a urine sample.

In some implementations of any of the above methods, the DNA duplex molecule comprises circulating-tumor DNA (ctDNA).

In some implementations of any of the above methods, the DNA duplex molecule comprises fetal cell-free DNA.

In some implementations of any of the above methods, the DNA duplex molecule is obtained from an individual with cancer or suspected of having cancer.

In some implementations of any of the above methods, the DNA duplex molecule is obtained from a transplant recipient.

Further described herein is a method of generating a DNA duplex overhang profile, comprising: generating a circular converted nucleic acid construct, comprising: directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule, to provide a circular nucleic acid construct; and converting (i) non-methylated cytosine residues in the circular nucleic acid construct into uracil residues, or (ii) methylated cytosine residues in the circular nucleic acid construct int uracil residues or dihydrouracil residues, wherein a duplex structure of the DNA duplex molecule is transformed into a single-stranded structure; amplifying the circular converted nucleic acid construct, thereby forming a linear converted nucleic acid construct, wherein the uracil residues or dihydrouracil residues in the circular converted nucleic acid construct are recognized as thymine residues in the amplifying; sequencing the linear converted nucleic acid construct, wherein the sequencing provides sequencing data; aligning, using one or more processors, to the sequencing data to a first converted nucleic acid reference sequence and a second converted nucleic acid reference sequence, wherein the first converted nucleic acid reference sequence and the second converted nucleic acid reference sequence are complements to each other when compared in an unconverted state; determining, using the one or more processors, a set of genomic coordinates within the first and second converted nucleic acid reference sequences, comprising a genomic coordinates for a first portion, a second portion, a third portion, and a fourth portion of the sequencing data; and detecting, using the one or more processors, based on the set of genomic coordinates, a presence or absence of a 3′ overhang for the first strand of the DNA duplex, a 5′ overhang for the first strand of the DNA duplex, a 3′ overhang for the second strand of the DNA duplex, and/or a 5′ overhang for the second strand of the DNA duplex.

In some implementations, the sequencing data comprising paired-end sequencing data comprising a first read and a second read; the aligning comprises aligning a first portion of the first read and a first portion of the second read to the first converted nucleic acid reference sequence, and aligning a second portion of the first read and a second portion of the second read to the second converted nucleic acid reference sequence, wherein the first converted nucleic acid reference sequence and the second converted nucleic acid reference sequence are complements to each other when compared in an unconverted state; and the set of genomic coordinates comprises a first genomic coordinate for the first portion of the first read, a second genomic coordinate for the first portion of the second read, a third genomic coordinate for the second portion of the first read, and a fourth genomic coordinate for the second portion of the second read.

In some implementations, the method further comprises determining, using the one or more processors, based on the set of coordinates, a 3′ overhang length for the first strand of the DNA duplex molecule, a 5′ overhang length for the first strand of the DNA duplex molecule, a 3′ overhang length for the second strand of the DNA duplex molecule, and/or a 5′ overhang length for the second strand of the DNA duplex molecule.

In some implementations, the method further comprises determining, using the one or more processors, a methylation status for one or more bases in the DNA duplex.

In some implementations, the method further comprises determining, using the one or more processors, a methylation status for one or more bases in a 3′ overhang or a 5′ overhang of the DNA duplex.

In some implementations, the DNA duple molecule is obtained from an individual, and the method further comprises comprising generating, using the one or more processors, a DNA duplex molecule overhang profile for the individual comprising information about overhangs for a plurality of DNA duplex molecules. In some implementations, the information comprises, for a plurality of DNA duplex molecules, the 3′ overhang length for the first strand of the DNA duplex molecule, a 5′ overhang length for the first strand of DNA duplex molecule, a 3′ overhang length for the second strand of the DNA duplex molecule, and/or a 5′ overhang length for the second strand of the DNA duplex molecule. In some implementations, the information comprises a sequence of a 5′ overhang or a 3′ overhang. In some implementations, the information comprises (1) a ratio, or a distribution of ratios, of the a 5′ overhang length for the first strand of DNA duplex molecule to a 3′ overhang length for the first strand of DNA duplex molecule, (2) a ratio, or a distribution of ratios, of the a 5′ overhang length for the first strand of DNA duplex molecule to a 3′ overhang length for the second strand of DNA duplex molecule, (3) a ratio, or a distribution of ratios, of the a 5′ overhang length for the first strand of DNA duplex molecule to a 5′ overhang length for the second strand of DNA duplex molecule, (4) a ratio, or a distribution of ratios, of the a 3′ overhang length for the first strand of DNA duplex molecule to a 5′ overhang length for the second strand of DNA duplex molecule, or (5) a ratio, or a distribution of ratios, of the a 3′ overhang length for the first strand of DNA duplex molecule to a 3′ overhang length for the second strand of DNA duplex molecule. In some implementations, the information comprises a ratio, or a distribution of ratios, of (1) a 5′ overhang length for the first strand of DNA duplex molecule, a 3′ overhang length for the first strand of DNA duplex molecule, a 5′ overhang length for the second strand of DNA duplex molecule, a 3′ overhang length for the second strand of DNA duplex molecule to (2) a duplex length.

In some implementations, the method further comprises comparing, using the one or more processors, the DNA duplex molecule overhang profile to a reference DNA duplex molecule overhang profile. In some implementations, the reference DNA duplex molecule overhang profile is based on DNA duplex molecules from a normal sample or a plurality of normal samples. In some implementations, the reference DNA duplex molecule overhang profile is based on DNA duplex molecules from a sample obtained from an individual with cancer or a plurality of individuals with cancer. In some implementations, the reference DNA duplex molecule overhang profile is based on DNA duplex molecules from a sample obtained from an individual with an abnormal fetus or a plurality of individuals with an abnormal fetus. In some implementations, the reference DNA duplex molecule overhang profile is based on DNA duplex molecules from a sample obtained from an individual that received a stable transplant or a plurality of individuals that received a stable transplant. In some implementations, the reference DNA duplex molecule overhang profile is based on DNA duplex molecules from a match normal sample obtained from the individual. In some implementations, the reference DNA duplex molecule overhang profile is based on DNA duplex molecules from a prior sample obtained from the individual.

In some implementations of the above method, the DNA duplex molecule is a cell-free DNA molecule.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A shows an exemplary method of making a nucleic acid construct from a nucleic acid duplex molecule, according to some embodiments.

FIG. 1B shows the exemplary method of FIG. 1A in a graphical representation, according to some embodiments.

FIG. 2 shows an exemplary linear converted nucleic acid construct along with a first read and second read obtained by sequencing the linear converted nucleic acid construct, according to some embodiments.

FIG. 3 shows an exemplary alignment of the first read and the second read to a first converted reference sequence and a second converted reference sequence, according to some embodiments.

FIG. 4 depicts an exemplary computing device or system in accordance with one embodiment of the present disclosure.

FIG. 5 depicts an exemplary computer system or computer network, in accordance with some instances of the systems described herein.

### DETAILED DESCRIPTION

A nucleic acid sequencing construct can be made from a DNA duplex molecule (e.g., cell-free DNA) to preserve the DNA molecular topology, including 3′ and/or 5′ overhangs, for sequencing and analysis. Thus, the methods described herein, among other things, allow for at least the determination of a length and/or sequence of a 3′ and/or 5′ overhang in a DNA duplex. Standard methods of assessing cfDNA do not adequately capture cfDNA 5′ and 3′ overhang information to provide complete topological information, which may provide information related to the onset, progression, diagnosis, treatment, etc. of various diseases, in particular for cancers. The loss of this information is an obstacle not only to the understanding of disease processes but also to the utilization of this information for the improvement of human health (e.g., early detection of diseases such as cancer, any association to treatment efficacies).

The nucleic acid sequencing construct also eliminates the need for duplex molecular barcode adapters to track both the positive and negative strands association with a particular double-stranded molecule through an assay. Duplex molecule barcode adapters, for example, have been used during sequence read analysis to reduce errors often produced by the sequencing process. Because both strands of the molecule are directly attached in the nucleic acid sequencing construction, duplex adapters are not needed to track the relationship between the positive and negative strands through the assay as they will be part of the same linear converted nucleic acid sequencing construct.

The methods described herein provide several distinct advantages over prior attempts to determine DNA duplex topology. For example, in some embodiments, the nucleic acid sequencing construct is made by directly attaching (e.g., ligating) ends of the first and second strands of the DNA duplex molecule to each other rather than through an adapter molecule (e.g., hairpin adapter) to provide adaptor-free circularization. Direct attachment of the first and second strands, rather than through an adapter molecule, may provide more efficient production of the nucleic acid construct (i.e., higher yield), which allows for increased recovery of DNA duplex molecule profile information as well as eliminating the need to track both strands of the original molecule (e.g., through the use of duplex molecule barcodes throughout the process (e.g., for use in reassociating the two strands from the original molecule during sequencing analysis (once they have been separate into single-stranded molecules). In another example, in some embodiments, the nucleic acid construct is made using inverse-PCR for amplification, as opposed to random primer extension techniques, which can provide more sensitive and/or target enriched nucleic acid constructs. Thus, using the methods described herein, it is possible to target specific subgenomic intervals, for example, when it is desired to obtain deep profiling coverage in selected regions of the genome.

In an exemplary implementation, a method of making a nucleic acid construct includes: directly attaching a 3′ end of a first strand of a DNA duplex molecule (e.g., a cell-free DNA duplex) to a 5′ end of a second strand of the DNA duplex molecule, and directly attaching a 3′ end of the second strand of the DNA duplex molecule to a 5′ end of the first strand of the DNA duplex molecule, to provide a circular nucleic acid construct; and converting (i) non-methylated cytosine residues in the circular nucleic acid construct into uracil residues, or (ii) methylated cytosine residues in the circular nucleic acid construct int uracil residues or dihydrouracil residues, wherein a duplex structure of the DNA duplex molecule is transformed into a single-stranded structure. Conversion at least partially disrupts the duplex structure of the DNA duplex molecule, resulting in a single-stranded circular nucleic acid construct. The converted circular nucleic acid construct can be amplified to form a linear converted nucleic acid construct, for example using inverse PCR. The amplification method may be a targeted amplification process, for example to enrich selected subgenomic intervals within a genome. Because the circular nucleic acid molecule is converted (i.e., by altering non-methylated or methylated cytosine residues), primers used in the amplification process may be specifically designed to hybridize to the converted sequence.

The linear converted nucleic acid sequencing construct may be sequenced, for example to provide a first read for a first strand of the linear converted nucleic acid construct and a second read for a second strand of the linear converted nucleic acid construct. The first read and the second read may each be paired-end sequence reads (i.e., the first read includes two paired reads and the second read includes two paired reads) or, alternatively the first read and the second read may be long-range reads encompassing the full first and second strands, respectively. The sequence reads can be analyzed to determine an overhang length and/or sequence of a 3′ and/or 5′ overhang of the first stand and/or second strand.

The length and/or sequence of the 3′ and/or 5′ overhang of the first stand and/or second strand may be used to determine a presence or absence of a disease, such as cancer. A DNA duplex topology profile (e.g., a DNA duplex molecule overhang profile) may be generated, either for the individual DNA duplex or a plurality of DNA duplexes. The profile may then be used to determine the presence or absence of the disease, for example by correlating the profile with a disease state profile.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, the term “subgenomic interval” (or “subgenomic sequence interval”) refers to a portion of a genomic sequence.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated.

Desirable effects of treatment include, but are not limited to, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

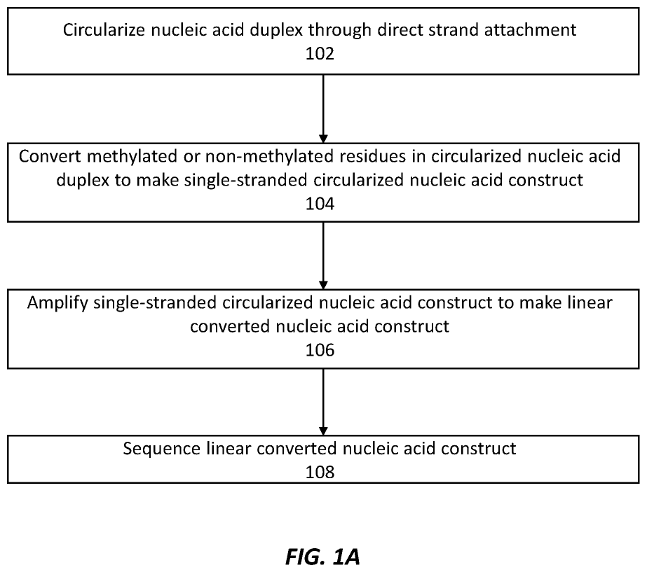

FIGS. 1-5 illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Nucleic Acid Construct

A nucleic acid construct, which may be used in accordance with the sequencing and/or methods described herein, can be derived from a nucleic acid duplex molecule (e.g., a DNA duplex molecule, such as a cell-free DNA duplex molecule). The DNA duplex molecule may be a naturally occurring DNA duplex molecule, which may be isolated according to the methods described herein. The DNA duplex molecule is then manipulated to provide the synthetic nucleic acid construct. Although the following discussion provides exemplary embodiments in reference to a DNA duplex molecule to generate a nucleic acid construct, it is understood that the disclosed methods may be applied to a plurality of DNA duplex molecules to provide a library comprising a plurality of nucleic acid constructs. For example, the method may include making a plurality of nucleic acid constructs in parallel or in the same reaction mixture.

The nucleic acid duplex molecule is circularized by directly attaching the ends of the first and second strands of the DNA duplex molecule to each other, for example through ligation. That is, the first and second strands may be concatenated to each other. The circularization of the DNA duplex molecule may be performed without the use of adapters (e.g., hairpin adapters). That is, the ends of the DNA duplex molecule may be directly attached to each other. For example, the circular nucleic acid construct may be provided by directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule.

The circular nucleic acid molecule may then be converted to transform a duplex structure of the nucleic acid duplex molecule into a single-stranded circular structure. In some embodiments, the conversion converts non-methylated cytosine residues into uracil residues. In some embodiments, the conversion converts methylated cytosine residues into uracil residues. In some embodiments, the conversion converts methylated cytosine residues into dihydrouracil residues. Conversion of cytosine residues into uracil or dihydrouracil residues disrupts base pairing between the first and second strands, thus causing the transformation of the duplex structure into a single-stranded structure. Since the first and second strands are attached at the ends of the strands, the resulting molecule has a single-stranded circular structure.

Direct attachment of the first and second strands to each other (i.e., without an intervening adapter sequence) provides the additional benefit of generating PCR amplification products that bridge the attached ends of the first and second strands without an intervening adapter sequence. As further discussed herein, an intervening adapter sequence would need to be sequenced without providing substantive information to determine the sequence at the ends of the first and second strands, which can degrade sequencing quality at the terminal ends of the sequence reads.

The nucleic acid sequencing construct also eliminates the need for duplex molecular barcode adapters to track both the positive and negative strands association with a particular double-stranded molecule through an assay. Duplex molecule barcode adapters, for example, have been used during sequence read analysis to reduce errors often produced by the sequencing process. Because both strands of the molecule are directly attached in the nucleic acid sequencing construction, duplex adapters are not needed to track the relationship between the positive and negative strands through the assay as they will be part of the same linear converted nucleic acid sequencing construct.

The converted circular nucleic acid construct may be amplified to form a linear converted nucleic acid construct. Uracil residues (or dihydrouracil residues) in the converted circular nucleic acid construct are recognized as thymine residues during the amplification process. In some implementations, amplification may be a single-stranded amplification (e.g., using a single amplification primer) process. Because the template is the circular nucleic acid construct the single-stranded amplification can produce a rolling circle amplification (RCA) product, which includes a plurality of concatenated converted first and second strands. In some implementations, the RCA product is itself amplified (for example, using polymerase chain reaction (PCR), which can create a double-stranded RCA product.

In some embodiments, the converted circular nucleic acid construct is amplified to form a linear converted nucleic acid construct using PCR to generate a double stranded PCR product. The PCR may be an inverse PCR, thus generating an inverse PCR product. See, for example, Ochman et al., Genetic Applications of an Inverse Polymerase Chain Reaction, Genetics, vol. 120, pp. 621-623 (1988). In some implementations, the inverse PCR product is a double-stranded linear nucleic acid construct that includes a first strand comprising a first converted sequence corresponding to the first strand of the original DNA duplex molecule and a second converted sequence corresponding to the second strand of the original DNA duplex molecule, although the first converted sequence is divided by the second converted sequence or, alternatively, the second converted sequence is divided by the first converted sequence. The inverse PCR product also includes a second strand complementary to the first strand.

The linear converted nucleic acid construct may be sequenced to generate sequencing reads, which may be further analyzed as described herein. The sequencing may be, for example, paired end sequencing.

FIG. 1A shows an exemplary method of making a nucleic acid construct from a nucleic acid duplex molecule, according to some embodiments. FIG. 1B shows the exemplary method in a graphical representation. The nucleic acid duplex molecule may be a DNA duplex molecule, such as a cell-free DNA molecule. In some implementations, the DNA duplex molecule is a genomic fragment (e.g., a fragment of a chromosome). The nucleic acid duplex molecule may be obtained (e.g., isolated) from a biological sample obtained from an individual.

At 102 of the method shown in FIG. 1, the nucleic acid duplex is circularized. To circularize the nucleic acid duplex, a 3′ end of a first strand of the duplex molecule is attached to a 5′ end of the second strand of the duplex molecule, and a 3′ end of the second strand of the duplex molecule is attached to a 5′ end of the first strand of the duplex molecule.

Attachment of the nucleic acid strands may be direct (i.e., without the use of an adapter or molecular barcode that joins the strands together). Accordingly, in some embodiments, the method includes directly attaching a 3′ end of a first strand of a DNA duplex molecule (e.g., cfDNA) to a 5′ end of a second strand of the DNA duplex molecule, and directly attaching a 3′ end of the second strand of the DNA duplex molecule to a 5′ end of the first strand of the DNA duplex molecule, to provide a circular nucleic acid construct.

Circularizing the duplex molecule may include, for example, phosphorylating the 5′ end of the first strand of the duplex molecule and the 5′ end of the second strand of duplex molecule. This process may be referred to as “end repair.” This process may be performed using a polynucleotide kinase. The polynucleotide kinase preferably lacks exonuclease activity (e.g., 5′→3′ exonuclease activity), thus avoiding any blunting of the ends of the duplex molecule. T4 polynucleotide kinase and Thermo PNK (available from A&A Biotechnology) are exemplary polynucleotide kinases that may be used for this process. A single-stranded ligase may be used to directly attach the 3′ end of the first strand of the duplex molecule to the 5′ end of the second strand of the duplex molecule, and directly attach the 3′ end of the second strand of the duplex molecule to the 5′ end of the first strand of the duplex molecule. Exemplary single-stranded ligases that may be used include CircLigase™ ssDNA Ligase (available from Lucigen) and CircLigase™ II ssDNA Ligase (available from Lucigen). See also Polidoros et al., Rolling circle amplification-RACE: a method for simultaneous isolation of 5′ and 3′ cDNA ends from amplified cDNA templates, BioTechniques, vol. 41, no. 1, pp. 35-40 (2006). Fraying the ends of the duplex molecule (while maintaining a partial duplex of the duplex molecule) may increase the efficiency of the single-stranded ligase activity and may be performed prior to directly attaching a 3′ end of a first strand of a duplex molecule to a 5′ end of a second strand of the DNA duplex molecule, and directly attaching a 3′ end of the second strand of the DNA duplex molecule to a 5′ end of the first strand of the duplex molecule. Fraying the ends of the duplex molecule may include, for example, heating the duplex molecule, chemically denaturing (for example, using sodium hydroxide (NaOH) or dimethyl sulfoxide (DMSO)) the duplex molecule, and/or combining a single-stranded DNA binding protein (SSB) with the duplex molecule.

At 104 non-methylated cytosine residues in the circular nucleic acid construct are converted into uracil residues, or, alternatively, methylated cytosine residues in the circular nucleic acid construct are converted into uracil residues or dihydrouracil residues. Converting the methylated or non-methylated cytosine residues in the circular nucleic acid construct causes the duplex structure of the duplex molecule to be transformed into a single-stranded structure. Conversion may be chemical or enzymatic. In some implementations, a bisulfite conversion reaction is used to convert non-methylated cytosine residues into uracil residues. For example, the construct may be contacted with a bisulfite compound, such as sodium bisulfite. Bisulfite leads to the deamination of unmethylated cytosine residues to uracil, leaving methylated cytosine residues intact. Alternatively, an enzymatic method may be used, for example by treating the nucleic acid construct with an enzyme that converts non-methylated cytosine to uracil, for example using NEBNext® Enzymatic Methyl-seq Kit (New England BioLabs), a ten-eleven translocation methylcytosine dioxygenase 2 (TET2) enzyme, or an APOBEC2 enzyme. Alternatively, methylated cytosine in the construct may be converted to uracil or dihydrouracil. See, for example, Liu et al., Bisulfate-free direct detection of 5-methylcytosine and 5-hydroxymethylcytosine at base resolution, Nature Biotechnology, vol. 37, pp. 424-429 (2019). Accordingly, in some implementations, methylated cytosine residues in the circular nucleic acid construct are converted into dihydrouracil residues or uracil residues using TET-assisted bisulfite treatment, or oxidative bisulfite treatment. See Yu et al., Base-Resolution Analysis of 5-Hydroxymethylcytosine in the Mammalian Genome, Cell, vol. 149, pp. 1368-1380 (2012); Yu et al., Tet-assisted bisulfite sequencing of 5-hydroxymethylcytosine, Nature Protocols, vol. 7, pp. 2159-2170 (2012); and Li et al., DNA Methylation Detection: Bisulfite Genomic Sequencing Analysis, Methods Mol. Biol., vol. 791, pp. 11-21 (2011); Booth et al., Quantitative sequencing of 5-methylcytosine and 5-hydroxymethylcytosine at single-base resolution, Science, vol. 336, no. 6083, pp. 934-937 (2012). The process of converting non-methylated cytosine to uracil (or, alternatively, methylated cytosine to uracil) results in a converted circular nucleic acid construct.

At 106 of FIG. 1A, the single-stranded circularized nucleic acid construct is amplified to make a linear converted nucleic acid construct. During the amplifying, uracil residues or dihydrouracil residues in the converted circular nucleic acid construct are recognized as thymine residues. That is, the amplification generates a complementary strand that includes adenine residues complementary to the uracil or dihydrouracil residues. In some implementations, the amplification is rolling circle amplification (RCA). The RCA process is a unidirectional amplification that includes the use of an amplification primer that is extended repeatedly through the converted circular nucleic acid construct, thereby providing a product with multiple concatenated copies of the first strand and the second strand (see FIG. 1B).

Alternatively, the amplification is a bidirectional amplification process, which generates a double-stranded polymerase chain reaction (PCR) product. In some implementations, the linear converted nucleic acid construct is an inverse PCR product, for example as shown in FIG. 1B. Optionally, primers used for amplification may include sequencing adaptor sequences (e.g., P5 and P7 sequencing adapter sequences), which is then incorporated into the resulting amplification product.

The amplification process may be used to specifically enrich one or more preselected subgenomic intervals within a genome. That is, the methods described herein need not generate whole genome libraries or analyze whole genomes. Instead, amplification primers may be designed to selectively amplify one or more targeted subgenomic intervals. Because the primers hybridize to converted nucleic acid molecules, the primer(s) may be designed to target converted sequences based on an expected converted sequence. See, for example, Tusnády et al., BiSearch: primer-design and search tool for PCR on bisulfite-treated genomes, Nucleic Acids Res., vol. 33, no. 1, e9(2005).

In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

The methods described herein can be used in combination with, or as part of, a method for evaluating a plurality or set of subject intervals (e.g., target sequences), e.g., from a set of genomic loci (e.g., gene loci or fragments thereof), as described herein. In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

In some instances, the disclosed methods may be used to selectively enrich for a gene or a portion of a gene selected from one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73,CDH1,CDK12, CDK4, CDK6, CDK8, CDKNIA, CDKN1B, CDKN2A, CDKN2B, CDKN2C, CEBPA, CHEK1, CHEK2, CIC, CREBBP, CRKL, CSF1R, CSF3R, CTCF, CTNNA1,CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, and ZNF703 or any combination thereof.

In some instances, the disclosed methods may be used to selectively enrich for a gene or a portion of a gene selected from one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-16, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

In some instances, the number of targeted subgenomic regions (i.e., different primers (for unidirectional amplification or primer pairs (for bidirectional amplification)) is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the amplification primer(s) may be designed to select a subgenomic region containing one or more rearrangements, e.g., an intron containing a genomic rearrangement. In such instances, the amplification primer(s) are designed such that repetitive sequences are masked to increase the selection efficiency.

Referring again to FIG. 1A, at 108, the linear converted nucleic acid construct is sequenced. Sequencing may occur through any suitable process. In some implementations of the method, sequencing is performed by next-generation sequencing. “Next-generation sequencing” (or “NGS”) as used herein may also be referred to as “massively parallel sequencing” (or “MPS”) and refers to any sequencing method that determines the nucleotide sequence of either individual nucleic acid molecules (e.g., as in single molecule sequencing) or clonally expanded proxies for individual nucleic acid molecules in a high throughput fashion. Next-generation sequencing methods are known in the art, and are described in, e.g., Metzker, M. (2010) Nature Biotechnology Reviews 11: 31-46, which is incorporated herein by reference. Other examples of sequencing methods suitable for use when implementing the methods and systems disclosed herein are described in, e.g., International Patent Application Publication No. WO 2012/092426. In some embodiments, the sequencing may comprise, for example, whole genome sequencing (WGS), whole exome sequencing, targeted sequencing or direct sequencing. In some embodiments, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., cfDNA 5′ and/or 3′ overhang sequences.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some embodiments, sequencing may comprise Illumina MiSeq sequencing. In some embodiments, sequencing may comprise Illumina HiSeq sequencing. In some embodiments, sequencing may comprise Illumina NovaSeq sequencing. Optimized methods for sequencing nucleic acids extracted from a sample are described in more detail in, e.g., International Patent Application Publication No. WO 2020/236941, the entire content of which is incorporated herein by reference.

### Determining Nucleic Acid Duplex Topology

Topological information for the nucleic acid molecule can be determined from sequencing reads generated by sequencing the nucleic acid construct made according to the methods described herein. For example, the sequence reads may be used to determine the length and/or sequence of a 5′ overhand and/or 3′ overhang of the nucleic acid duplex molecule. The sequencing data generated by sequencing the linear converted nucleic acid construct can include paired-end sequence reads (i.e., a first sequence read for the first strand of the linear converted nucleic acid construct and a second read for a second strand of the linear converted nucleic acid construct).

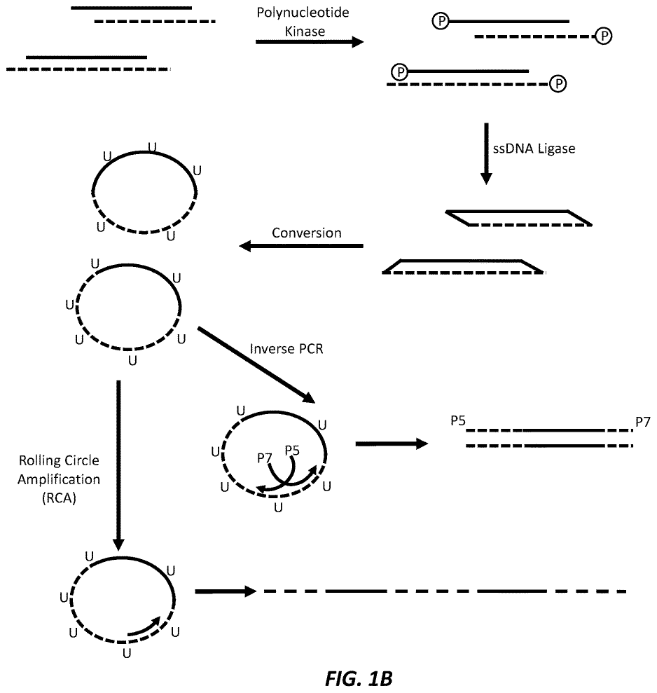

In some embodiments, the sequence read(s) include a portion of a sequence from the first strand of the original nucleic acid duplex molecule and a portion of a sequence from the second strand of the original nucleic acid duplex molecule, including a junction where the first strand was directly attached to the second strand. FIG. 2 shows an exemplary linear converted nucleic acid construct made as discussed herein (see, for example, FIG. 1B) along with a first read and second read obtained by sequencing the linear converted nucleic acid construct made as discussed herein. In the example shown in FIG. 2, the solid line of the linear converted nucleic acid construct is derived from the first strand of the starting nucleic acid duplex molecule, and the dashed line of the linear converted nucleic acid construct is derived from the second strand of the starting nucleic acid duplex molecule. The exemplary linear converted nucleic acid construct was generated using inverse PCR primers that targeted the second strand of the converted circularized nucleic acid construct. FIG. 2 describes a paired end sequencing process, but as those skilled in the art will appreciate, other sequencing techniques may be used. Regardless, in FIG. 2, the first read includes, in order, a portion of the converted sequence corresponding to the second strand of the starting nucleic acid molecule, a junction, and a portion of the converted sequence corresponding to the first strand of the starting nucleic acid molecule. The second read also includes, in order, a portion of the converted sequence corresponding to the second strand of the starting nucleic acid molecule, a junction, and a portion of the converted sequence corresponding to the first strand of the starting nucleic acid molecule. However, the portion corresponding to the second strand of the starting nucleic acid molecule in the first read differs from the portion corresponding to the second strand of the starting nucleic acid molecule in the second read.

The sequencing data (including the first read and the second read) can be analyzed to detect the junctions in the sequencing reads and, based on the sequencing data, determine the length and/or sequence of a 5′ overhand and/or 3′ overhang of the nucleic acid duplex molecule.

In some implementations the first read and second read are aligned to a reference sequence. The reference sequence is a converted reference sequence that has been converted through the same process used to generate the linear converted nucleic acid sequencing construct. Alignment is the process of matching a query sequence read with one or more additional sequence reads or reference sequence. Alignment may further include mapping of the sequence read, for example to a location, e.g., a genomic location or locus, within a reference sequence. In some embodiments, a sequence reads may be aligned to a known reference sequence (e.g., a wild-type sequence). In some embodiments, the reference sequences can be obtained from databases of the human genome (e.g., the HG19 human reference genome) or cancer mutations (e.g., COSMIC). Methods of sequence alignment for sequence reads are described in, e.g., Trapnell, C. and Salzberg, S. L. Nature Biotech., 2009, 27: 455-457. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Examples of alignment tools optimized for aligning sequence reads for converted DNA include, but are not limited to, NovoAlign (Novocraft Technologies, Selangor, Malaysia), and the Bismark tool (Krueger et al., Bismark: A Flexible Aligner and Methylation Caller for Bisulfite-Seq Applications, Bioinformatics, vol. 27, no. 11, pp. 1571-1572 (2011)). In some embodiments, the first read and the second read can also be merged to generate one or more merged sequence reads representing the first and second strands of the nucleic acid molecule.

In some embodiments, the methods and systems disclosed herein may integrate the use of multiple, individually tuned, alignment methods or algorithms to optimize base-calling performance in sequencing methods, particularly in methods that rely on massively parallel sequencing (MPS). In some embodiments, the disclosed methods and systems may comprise the use of one or more global alignment algorithms. In some embodiments, the disclosed methods and systems may comprise the use of one or more local alignment algorithms.

Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25: 1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1): 195-197), the Striped Smith-Waterman algorithm (see, e.g., Farrar (2007), “Striped Smith-Waterman Speeds Database Searches Six Times Over Other SIMD Implementations”, Bioinformatics 23(2): 156-161), the Needleman-Wunsch algorithm (Needleman, et al. (1970) “A General Method Applicable to the Search for Similarities in the Amino Acid Sequence of Two Proteins”, J. Molecular Biology 48(3): 443-53), or any combination thereof.

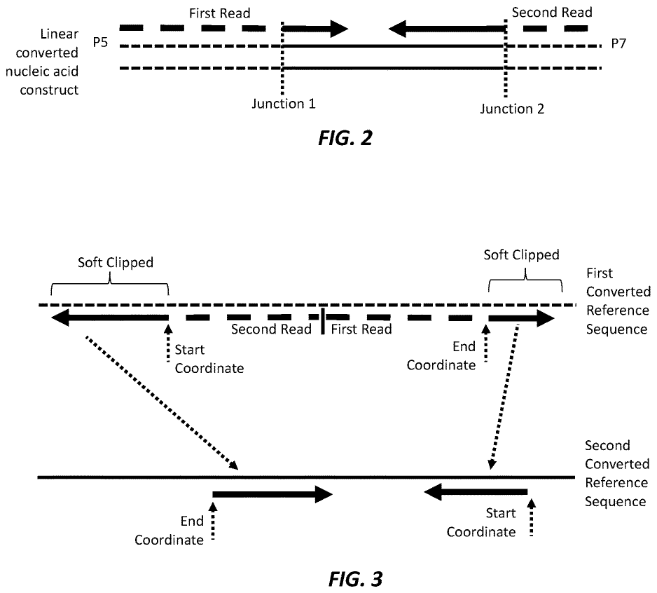

In some implementations, the first read and the second read are aligned to a first converted reference sequence and a second converted reference sequence, wherein the first converted nucleic acid reference sequence and the second converted nucleic acid reference sequence are complements to each other when compared in an unconverted state. FIG. 3 shows an exemplary alignment of the first read and the second read (as shown in FIG. 2) to a first converted reference sequence and a second converted reference sequence. It should be noted that, although FIG. 2 and FIG. 3 depict sequence and alignment methods that utilize a paired-end sequencing process, other sequencing methods can be used that do not utilize paired-end sequencing. Regardless, in the exemplary approach shown in FIG. 3, the first converted reference sequence corresponds to the same genomic strand as the second strand of the original nucleic acid duplex (as indicated by the dashed line). The portions of the first read and the second read that align with the first converted reference sequence provide coordinates (i.e., start coordinate (e.g., 5′ coordinate) and end coordinate (e.g., 3′ coordinate)) within the reference sequence for the second strand of the original nucleic acid duplex. The portions of the first read and the second read that do not align with the first converted reference sequence can be soft clipped. The soft clipped portions of the first read and second read can then be aligned to a second converted reference sequence (as indicated in FIG. 3 by a solid line), which is a complement to the first converted reference sequences when each sequence is in an unconverted state. The soft clipped portions of the first read and the second read that did not align with the first converted reference can be aligned to the second converted reference sequence, which provides coordinates (i.e., start coordinate (e.g., 5′ coordinate) and end coordinate (e.g., 3′ coordinate)) within the second reference sequence for the first strand of the original nucleic acid duplex. Thus, alignment of all four coordinates of the two reads (i.e., a first portion aligned to a first converted sequence and a soft-clipped portion aligned to the second converted reference sequence, for each read) can be determined through the alignment.

With the 5′ and 3′ coordinates of the first strand and the second strand of the starting nucleic acid duplex molecule determined, a presence or absence of a 3′ overhang for the first strand of the duplex, a 5′ overhang for the first strand of the duplex, a 3′ overhang for the second strand of the duplex, and/or a 5′ overhang for the second strand of the duplex is, in some embodiments, detected based on the set of coordinates. The coordinates may also be analyzed, in some embodiments, to determine a 3′ overhang length for the first strand of the duplex molecule, a 5′ overhang length for the first strand of the duplex molecule, a 3′ overhang length for the second strand of the duplex molecule, and/or a 5′ overhang length for the second strand of the duplex molecule. For example, the difference between a start coordinate (e.g., 5′ coordinate) in the first strand of the duplex molecule and an end coordinate (e.g., 3′ coordinate) in the second strand of the duplex molecule can provide a length of a 5′ overhang in the first strand or a 3′ overhang in the second strand. In another example, the difference between an end coordinate (e.g., 3′ coordinate) in the first strand of the duplex molecule and a start coordinate (e.g., 5′ coordinate) in the second strand of the duplex molecule can provide a length of a 3′ overhang in the first strand or a 5′ overhang in the second strand. In some embodiments, the coordinates are analyzed to determine a 3′ overhang sequence for the first strand of the duplex molecule, a 5′ overhang sequence for the first strand of the duplex molecule, a 3′ overhang sequence for the second strand of the duplex molecule, and/or a 5′ overhang sequence for the second strand of the duplex molecule. For example, the length of the 5′ and/or 3′ overhang in the first strand or the second strand may be compared to the sequencing data to determine the sequence of the corresponding overhang.

In some embodiments, the method may include determining a methylation status for one or more bases in the duplex molecule, for example in the 5′ and/or 3′ overhang in the first strand or the second strand.

In some implementations of any of the methods described, herein, the DNA duplex molecule is obtained from an individual. The method can further include generating a DNA duplex molecule overhang profile for the individual comprising information about overhangs for a plurality of DNA duplex molecules. For example, the information can include, for a plurality of DNA duplex molecules, the 3′ overhang length for the first strand of the DNA duplex molecule, a 5′ overhang length for the first strand of DNA duplex molecule, a 3′ overhang length for the second strand of the DNA duplex molecule, and/or a 5′ overhang length for the second strand of the DNA duplex molecule. In some implementations, the information comprises a sequence of a 5′ overhang or a 3′ overhang. In some implementations, the information comprises (1) a ratio of the a 5′ overhang length for the first strand of DNA duplex molecule to a 3′ overhang length for the first strand of DNA duplex molecule, (2) a ratio of the a 5′ overhang length for the first strand of DNA duplex molecule to a 3′ overhang length for the second strand of DNA duplex molecule, (3) a ratio of the a 5′ overhang length for the first strand of DNA duplex molecule to a 5′ overhang length for the second strand of DNA duplex molecule, (4) a ratio of the a 3′ overhang length for the first strand of DNA duplex molecule to a 5′ overhang length for the second strand of DNA duplex molecule, or (5) a ratio of the a 3′ overhang length for the first strand of DNA duplex molecule to a 3′ overhang length for the second strand of DNA duplex molecule. In some implementations, the information comprises a ratio of (1) a 5′ overhang length for the first strand of DNA duplex molecule, a 3′ overhang length for the first strand of DNA duplex molecule, a 5′ overhang length for the second strand of DNA duplex molecule, a 3′ overhang length for the second strand of DNA duplex molecule to (2) a duplex length. In some implementations, the information comprises a distribution of any of the above (e.g., a distribution of ratios). The distribution may be a distribution for a plurality of duplex DNA molecules.

The method may further include comparing, for example using one or more processors, the DNA duplex molecule overhang profile to a reference DNA duplex molecule overhang profile. In some implementations, the reference DNA duplex molecule overhang profile is based on DNA duplex molecules from a normal sample or a plurality of normal samples. In some implementations, the reference DNA duplex molecule overhang profile is based on DNA duplex molecules from a sample obtained from an individual with cancer or a plurality of individuals with cancer. In some implementations, the reference DNA duplex molecule overhang profile is based on DNA duplex molecules from a sample obtained from an individual with an abnormal fetus or a plurality of individuals with an abnormal fetus. In some implementations, the reference DNA duplex molecule overhang profile is based on DNA duplex molecules from a sample obtained from an individual that received a stable transplant or a plurality of individuals that received a stable transplant. In some implementations, the reference DNA duplex molecule overhang profile is based on DNA duplex molecules from a match normal sample obtained from the individual. In some implementations, the reference DNA duplex molecule overhang profile is based on DNA duplex molecules from a prior sample obtained from the individual.

In some embodiments, the method described herein further comprises generating, by one or more processors, a report indicating the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex, or other DNA duplex molecule overhang profile information. In some embodiments, the method further comprises transmitting the report to a healthcare provider. In some embodiments, the report is transmitted via a computer network or a peer-to-peer connection.

In some embodiments, the method further comprises generating a genomic profile for the subject comprising the length of the 3′ overhang of the first strand of the cell-free DNA duplex, the 3′ overhang of the second strand of the cell-free DNA duplex, the 5′ overhang of the first strand of the cell-free DNA duplex, and/or the 5′ overhang of the second strand of the cell-free DNA duplex. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the subject further comprises results from a nucleic acid sequencing-based test.

The DNA topological information, such as 3′ overhang and/or 5′ overhang length and/or sequence information determined according to the methods described herein, may be used to detect the presence or absence of a disease, for example, cancer. This is particularly useful, for example, in determining the presence or absence of disease in an early-stage cancer patient or a cancer patient having low cancer levels. For example, in some implementations, the individual has a circulating tumor DNA (ctDNA) content of less than 1%, less than 0.8%, less than 0.5%, less than 0.3%, less than 0.1%, or less than 0.05%.

### Samples

The disclosed methods and systems may be used with any of a variety of samples (also referred to herein as specimens) comprising nucleic acids (e.g., DNA) that are collected from a subject (e.g., a patient). Examples of a sample include, but are not limited to, a liquid biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or sample derived therefrom).

In some embodiments, the sample may be collected by needle biopsy, fine needle aspiration, collection cup or tube, oral swab, nasal swab, vaginal swab or a cytology smear, etc.

In some embodiments, the sample is a liquid biopsy sample, and may comprise, e.g., whole blood, blood plasma, blood serum, urine, stool, sputum, saliva, or cerebrospinal fluid. In some embodiments, the sample may be a liquid biopsy sample and may comprise circulating tumor cells (CTCs). In some embodiments, the sample may be a liquid biopsy sample and may comprise cell-free DNA (cfDNA), circulating tumor DNA (ctDNA), or any combination thereof.

In some embodiments, the disclosed methods may further comprise analyzing a primary control (e.g., a normal blood sample). In some embodiments, the disclosed methods may further comprise determining if a primary control is available and, if so, isolating a control nucleic acid (e.g., DNA) from said primary control. In some embodiments, the sample may comprise any normal control if no primary control is available. In some embodiments, the method includes evaluating a sample, e.g., a normal sample using the methods described herein. In some embodiments, the disclosed methods may further comprise determining that no primary control is available, and marking said sample for analysis without a matched control.

In some embodiments, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some embodiments, cell-free or circulating tumor DNA is extracted from the liquid sample. In some embodiments, a sample with low nucleated cellularity may require more, e.g., greater volume for DNA extraction.

In some embodiments, the method for determining cell-free DNA topology further comprises obtaining the cell-free DNA duplex from a subject. In some embodiments, the cell-free DNA duplex is obtained from a liquid biopsy sample. In some embodiments, the sample is a liquid biopsy sample and comprises blood, plasma, cerebrospinal fluid, sputum, stool, urine, or saliva. In some embodiments, the cell-free DNA duplex is a circulating tumor DNA (ctDNA) duplex.

### Subjects

In some embodiments, the sample is obtained (e.g., collected) from a subject (e.g., a human patient) with a condition or disease (e.g., a hyperproliferative disease (such as cancer) or a non-cancer indication) or suspected of having the condition or disease. In some embodiments, the hyperproliferative disease is a cancer. In some embodiments, the cancer is a solid tumor or a metastatic form thereof. In some embodiments, the cancer is a hematological cancer, e.g., a leukemia or lymphoma.

In some embodiments, the subject has a cancer or is at risk of having a cancer. For example, in some embodiments, the subject has a genetic predisposition to a cancer (e.g., having a genetic mutation that increases their baseline risk for developing a cancer). In some embodiments, the subject has been exposed to an environmental perturbation (e.g., radiation or a chemical) that increases their risk for developing a cancer. In some embodiments, the subject is in need of being monitored for development of a cancer. In some embodiments, the subject is in need of being monitored for cancer progression or regression, e.g., after being treated with an anti-cancer therapy (or anti-cancer treatment). In some embodiments, the subject is in need of being monitored for relapse of cancer. In some embodiments, the subject is in need of being monitored for minimum residual disease (MRD). In some embodiments, the subject has been or is being treated for cancer. In some embodiments, the subject has not been treated with an anti-cancer therapy (or anti-cancer treatment).

In some embodiments, the subject (e.g., a patient) is being treated, or has been previously treated, with one or more anti-cancer therapies. In some embodiments, e.g., for a patient who has been previously treated with a targeted anti-cancer therapy, a post-targeted therapy sample (e.g., specimen) is obtained (e.g., collected). In some embodiments, the post-targeted therapy sample is a sample obtained after the completion of the targeted therapy. In some embodiments, the one or more anti-cancer therapies (or anti-cancer treatments) can include, but are not limited to, surgery (e.g., surgical resection), radiation therapy, or chemotherapy and mixtures thereof or the like.

In some embodiments, the patient has not been previously treated with an anti-cancer therapy. In some embodiments, e.g., for a patient who has not been previously treated with a targeted anti-cancer therapy, the sample comprises a liquid biopsy, e.g., an original liquid biopsy, or a liquid biopsy following recurrence.

In some embodiments, the DNA duplex molecule is obtained from an individual with cancer or suspected of having cancer. In some embodiments, the DNA duplex molecule is obtained from a transplant recipient. In some embodiments, the DNA duplex is obtained from a pregnant mother.

In some embodiments, the method for determining cell-free DNA topology further comprises obtaining the cell-free DNA duplex from a subject. In some embodiments, the cell-free DNA duplex is obtained from a subject suspected of having cancer or determined to have cancer. In some embodiments, the cell-free DNA duplex is obtained from a liquid biopsy sample. In some embodiments, the sample is a liquid biopsy sample and comprises blood, plasma, cerebrospinal fluid, sputum, stool, urine, or saliva. In some embodiments, the cell-free DNA duplex is a circulating tumor DNA (ctDNA) duplex. In some embodiments, the method further comprises treating the subject with an anti-cancer therapy.

### Nucleic Acid Extraction and Processing

DNA may be extracted from liquid biopsy samples, including but not limited to blood, plasma, cerebrospinal fluid, sputum, stool, urine, or saliva or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009: 574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM 333, Jan. 1, 2011, Promega Corporation, Madison, WI)).

A typical DNA extraction procedure, for example, comprises (i) collection of the fluid sample from which DNA is to be extracted, (ii) treatment of the fluid sample with a concentrated salt solution to precipitate proteins, lipids, and RNA, followed by centrifugation to separate out the precipitated proteins, lipids, and RNA, and (iii) purification of DNA from the supernatant to remove detergents, proteins, salts, or other reagents used during previous steps. The DNA sample optionally may be further treated with an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water.

### Systems and Storage Media

Also disclosed herein are systems designed to implement any of the disclosed methods for generating a nucleic acid duplex topology (e.g., overhang) profile. The systems may comprise, e.g., one or more processors, and a memory unit communicatively coupled to the one or more processors and configured to store instructions that, when executed by the one or more processors, cause the system to: align, using one or more processors, a first portion of the first read and a first portion of the second read to a first converted nucleic acid reference sequence, and aligning a second portion of the first read and a second portion of the second read to second converted nucleic acid reference sequence, wherein the first converted nucleic acid reference sequence and the second converted nucleic acid reference sequence are complements to each other when compared in an unconverted state; determine, using the one or more processors, a set of genomic coordinates within the first and second converted nucleic acid reference sequences, comprising a first genomic coordinate for the first portion of the first read, a second genomic coordinate for the first portion of the second read, a third genomic coordinate for the second portion of the first read, and a fourth genomic coordinate for the second portion of the second read; and detect, using the one or more processors, based on the set of genomic coordinates, a presence or absence of a 3′ overhang for the first strand of the DNA duplex, a 5′ overhang for the first strand of the DNA duplex, a 3′ overhang for the second strand of the DNA duplex, and/or a 5′ overhang for the second strand of the DNA duplex.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

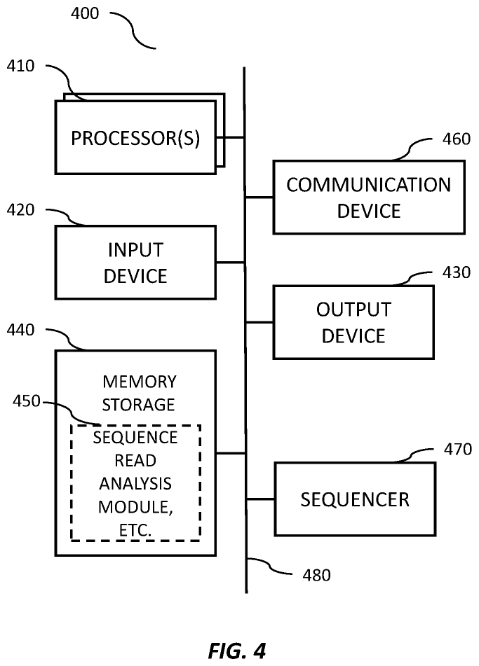

FIG. 4 illustrates an example of a computing device or system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage device(s) 440, communication devices 460, and nucleic acid sequencer(s) 470. Software module (450 residing in memory or storage device 440 may comprise, e.g., an operating system as well as software for executing the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage device 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 640 and executed by processor(s) 410, can include, for example, an operating system and/or the processes that embody the functionality of the methods of the present disclosure (e.g., as embodied in the devices as described herein).

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage device 440, that can contain or store processes for use by or in connection with an instruction execution system, apparatus, or device.

Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

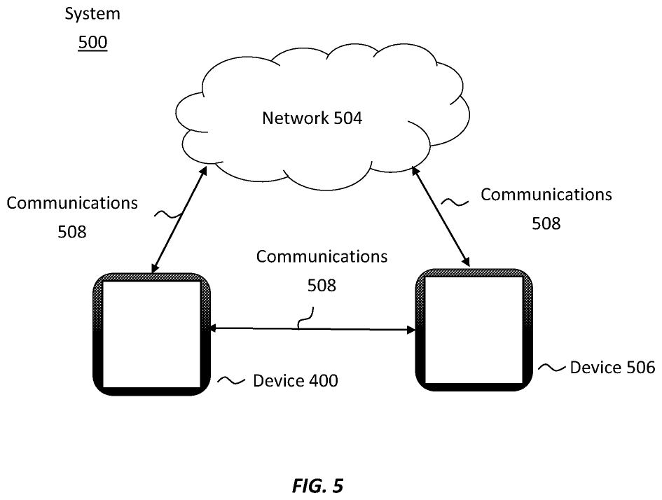

Device 400 may be connected to a network (e.g., network 504, as shown in FIG. 5 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can be implemented using any operating system, e.g., an operating system suitable for operating on the network. Software module 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 410.

Device 400 can further include a sequencer 470, which can be any suitable nucleic acid sequencing instrument.

FIG. 5 illustrates an example of a computing system in accordance with one embodiment. In system 500, device 400 (e.g., as described above and illustrated in FIG. 4) is connected to network 504, which is also connected to device 506. In some embodiments, device 506 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 400 and 506 may communicate, e.g., using suitable communication interfaces via network 504, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, Devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 508, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### Exemplary Embodiments

The following embodiments are exemplary and are not intended to limit the scope of any invention described herein. Among the exemplary embodiments include:
Embodiment 1. A method of making a nucleic acid construct comprising:
directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule, to provide a circular nucleic acid construct; and
converting (i) non-methylated cytosine residues in the circular nucleic acid construct into uracil residues, or (ii) methylated cytosine residues in the circular nucleic acid construct into uracil residues or dihydrouracil residues, wherein a duplex structure of the DNA duplex molecule is transformed into a single-stranded structure.
Embodiment 2. The method of embodiment 1, wherein the DNA duplex molecule is a cell-free DNA duplex molecule.
Embodiment 3. The method of embodiment 1 or 2, comprising phosphorylating the 5′ end of the first strand of the DNA duplex molecule and the 5′ end of the second strand of DNA duplex molecule.
Embodiment 4. The method of embodiment 3, wherein the 5′ end of the first strand of the DNA duplex molecule and the 5′ end of the second strand of the DNA duplex molecule are phosphorylated using a T4 polynucleotide kinase.
Embodiment 5. The method of any one of embodiments 1-4, wherein the 3′ end of the first strand of the DNA duplex molecule is directly attached to the 5′ end of the second strand of the DNA duplex molecule, and the 3′ end of the second strand of the DNA duplex molecule is directly attached to the 5′ end of the first strand of the DNA duplex molecule, using a single-stranded DNA (ssDNA) ligase.
Embodiment 6. The method of any one of embodiments 1-5, comprising fraying the ends of the DNA duplex molecule while maintaining a partial duplex of the DNA duplex molecule prior to directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule.
Embodiment 7. The method of embodiment 6, wherein the fraying comprises heating the DNA duplex molecule or chemically denaturing the DNA duplex molecule.
Embodiment 8. The method of any one of embodiments 1-7, wherein non-methylated cytosine residues in the circular nucleic acid construct are converted into uracil residues using a bisulfite reaction.
Embodiment 9. The method of any one of embodiments 1-8, wherein non-methylated cytosine residues in the circular nucleic acid construct are converted into uracil residues using an enzymatic reaction.
Embodiment 10. The method of any one of embodiments 1-9, wherein methylated cytosine residues in the circular nucleic acid construct are converted into dihydrouracil residues or uracil residues using TET-assisted bisulfite treatment or oxidative bisulfite treatment.
Embodiment 11. The method of any one of embodiments 1-10, amplifying the circular converted nucleic acid construct, thereby forming a linear converted nucleic acid construct, wherein the uracil residues or dihydrouracil residues in the circular converted nucleic acid construct are recognized as thymine residues in the amplifying.
Embodiment 12. The method of embodiment 11, wherein the amplifying targets a subgenomic interval within a genome.
Embodiment 13. The method of embodiment 11 or 12, wherein the linear converted nucleic acid construct is a rolling circle amplification (RCA) product.
Embodiment 14. The method of embodiment 11 or 12, wherein the linear converted nucleic acid construct is a double-stranded polymerase chain reaction (PCR) product.
Embodiment 15. The method of embodiment 14, wherein the linear converted nucleic acid construct is a double-stranded inverse PCR product.
Embodiment 16. The method of embodiment 14 or 15, wherein the amplifying comprises using a first primer targeting a converted first sequence and a second primer targeting a converted second sequence.
Embodiment 17. A method comprising sequencing the linear converted nucleic acid construct made according to the method of any one of embodiments 11-16 to provide sequencing data.
Embodiment 18. The method of embodiment 17, wherein the sequencing data comprises paired-end sequencing data comprising a first and a second read.
Embodiment 19. The method of embodiment 18, further comprising aligning a first portion of the first read and a first portion of the second read to a first converted nucleic acid reference sequence, and aligning a second portion of the first read and a second portion of the second read to second converted nucleic acid reference sequence, wherein the first converted nucleic acid reference sequence and the second converted nucleic acid reference sequence are complements to each other when compared in an unconverted state.
Embodiment 20. The method of embodiment 19, further comprising determining a set of genomic coordinates within the first and second converted nucleic acid reference sequences, comprising a first genomic coordinate for the first portion of the first read, a second genomic coordinate for the first portion of the second read, a third genomic coordinate for the second portion of the first read, and a fourth genomic coordinate for the second portion of the second read.
Embodiment 21. The method of embodiment 20, further comprising determining, based on the set of genomic coordinates, a presence or absence of a 3′ overhang for the first strand of the DNA duplex, a 5′ overhang for the first strand of the DNA duplex, a 3′ overhang for the second strand of the DNA duplex, and/or a 5′ overhang for the second strand of the DNA duplex.
Embodiment 22. The method of embodiment 20 or 21, further comprising determining, based on the set of coordinates, a 3′ overhang length for the first strand of the DNA duplex molecule, a 5′ overhang length for the first strand of the DNA duplex molecule, a 3′ overhang length for the second strand of the DNA duplex molecule, and/or a 5′ overhang length for the second strand of the DNA duplex molecule.
Embodiment 23. The method of any one of embodiments 17-22, comprising determining a methylation status for one or more bases in the DNA duplex molecule.
Embodiment 24. The method of embodiment 23, comprising determining a methylation status for one or more bases in a 3′ overhang or a 5′ overhang of the DNA duplex molecule.
Embodiment 25. The method of any one of embodiments 17-24, wherein the DNA duplex molecule is obtained from an individual, and the method further comprises comprising generating a DNA duplex molecule overhang profile for the individual comprising information about overhangs for a plurality of DNA duplex molecules.
Embodiment 26. The method of embodiment 25, wherein the information comprises, for a plurality of DNA duplex molecules, the 3′ overhang length for the first strand of the DNA duplex molecule, a 5′ overhang length for the first strand of DNA duplex molecule, a 3′ overhang length for the second strand of the DNA duplex molecule, and/or a 5′ overhang length for the second strand of the DNA duplex molecule.
Embodiment 27. The method of embodiment 25 or 26, wherein the information comprises a sequence of a 5′ overhang or a 3′ overhang.
Embodiment 28. The method of any one of embodiments 25-27, wherein the information comprises (1) a ratio, or a distribution of ratios, of the a 5′ overhang length for the first strand of DNA duplex molecule to a 3′ overhang length for the first strand of DNA duplex molecule, (2) a ratio, or a distribution of ratios, of the a 5′ overhang length for the first strand of DNA duplex molecule to a 3′ overhang length for the second strand of DNA duplex molecule, (3) a ratio, or a distribution of ratios, of the a 5′ overhang length for the first strand of DNA duplex molecule to a 5′ overhang length for the second strand of DNA duplex molecule, (4) a ratio, or a distribution of ratios, of the a 3′ overhang length for the first strand of DNA duplex molecule to a 5′ overhang length for the second strand of DNA duplex molecule, or (5) a ratio, or a distribution or ratios, of the a 3′ overhang length for the first strand of DNA duplex molecule to a 3′ overhang length for the second strand of DNA duplex molecule.
Embodiment 29. The method of any one of embodiments 25-28, wherein the information comprises a ratio of (1) a 5′ overhang length for the first strand of DNA duplex molecule, a 3′ overhang length for the first strand of DNA duplex molecule, a 5′ overhang length for the second strand of DNA duplex molecule, a 3′ overhang length for the second strand of DNA duplex molecule to (2) a duplex length.
Embodiment 30. The method of any one of embodiments 25-29, further comprising comparing the DNA duplex molecule overhang profile to a reference DNA duplex molecule overhang profile.
Embodiment 31. The method of embodiment 30, wherein the reference DNA duplex molecule overhang profile is based on DNA duplex molecules from a normal sample or a plurality of normal samples.
Embodiment 32. The method of embodiment 31, wherein the reference DNA duplex molecule overhang profile is based on DNA duplex molecules from a normal sample, wherein the normal sample is synthetically created from a plurality of individuals.
Embodiment 33. The method of embodiment 30, wherein the reference DNA duplex molecule overhang profile is based on DNA duplex molecules from a sample obtained from an individual with cancer or a plurality of individuals with cancer.
Embodiment 34. The method of embodiment 30, wherein the reference DNA duplex molecule overhang profile is based on DNA duplex molecules from a sample obtained from an individual with an abnormal fetus or a plurality of individuals with an abnormal fetus.
Embodiment 35. The method of embodiment 30, wherein the reference DNA duplex molecule overhang profile is based on DNA duplex molecules from a sample obtained from an individual that received a stable transplant or a plurality of individuals that received a stable transplant.
Embodiment 36. The method of embodiment 30, wherein the reference DNA duplex molecule overhang profile is based on DNA duplex molecules from a match normal sample obtained from the individual.
Embodiment 37. The method of embodiment 30, wherein the reference DNA duplex molecule overhang profile is based on DNA duplex molecules from a prior sample obtained from the individual.
Embodiment 38. The method of any one of embodiments 1-37, wherein the DNA duplex molecule is obtained from a blood, plasma, serum, saliva, pleural fluid, or cerebrospinal fluid sample.
Embodiment 39. The method of any one of embodiments 1-38, wherein the DNA duplex molecule is obtained from a urine sample.
Embodiment 40. The method of any one of embodiments 1-39, wherein the DNA duplex molecule comprises circulating-tumor DNA (ctDNA).
Embodiment 41. The method of any one of embodiments 1-39, wherein the DNA duplex molecule comprises fetal cell-free DNA.
Embodiment 42. The method of any one of embodiments 1-39, wherein the DNA duplex molecule is obtained from an individual with cancer or suspected of having cancer.
Embodiment 43. The method of any one of embodiments 1-39, wherein the DNA duplex molecule is obtained from a transplant recipient.
Embodiment 44. A nucleic acid construct made according to the method of any one of embodiments 1-16.
Embodiment 45. A method of generating a DNA duplex overhang profile, comprising:
generating a circular converted nucleic acid construct, comprising:
directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule, to provide a circular nucleic acid construct; and
converting (i) non-methylated cytosine residues in the circular nucleic acid construct into uracil residues, or (ii) methylated cytosine residues in the circular nucleic acid construct int uracil residues or dihydrouracil residues, wherein a duplex structure of the DNA duplex molecule is transformed into a single-stranded structure;
amplifying the circular converted nucleic acid construct, thereby forming a linear converted nucleic acid construct, wherein the uracil residues or dihydrouracil residues in the circular converted nucleic acid construct are recognized as thymine residues in the amplifying;
sequencing the linear converted nucleic acid construct, wherein the sequencing provides sequencing data;
aligning, using one or more processors, to the sequencing data to a first converted nucleic acid reference sequence and a second converted nucleic acid reference sequence, wherein the first converted nucleic acid reference sequence and the second converted nucleic acid reference sequence are complements to each other when compared in an unconverted state;
determining, using the one or more processors, a set of genomic coordinates within the first and second converted nucleic acid reference sequences, comprising a genomic coordinates for a first portion, a second portion, a third portion, and a fourth portion of the sequencing data; and
detecting, using the one or more processors, based on the set of genomic coordinates, a presence or absence of a 3′ overhang for the first strand of the DNA duplex, a 5′ overhang for the first strand of the DNA duplex, a 3′ overhang for the second strand of the DNA duplex, and/or a 5′ overhang for the second strand of the DNA duplex.
Embodiment 46. The method of embodiment 45, wherein:
the sequencing data comprising paired-end sequencing data comprising a first read and a second read;
the aligning comprises aligning a first portion of the first read and a first portion of the second read to the first converted nucleic acid reference sequence, and aligning a second portion of the first read and a second portion of the second read to the second converted nucleic acid reference sequence, wherein the first converted nucleic acid reference sequence and the second converted nucleic acid reference sequence are complements to each other when compared in an unconverted state; and
the set of genomic coordinates comprises a first genomic coordinate for the first portion of the first read, a second genomic coordinate for the first portion of the second read, a third genomic coordinate for the second portion of the first read, and a fourth genomic coordinate for the second portion of the second read.
Embodiment 47. The method of embodiment 45 or 46, further comprising determining, using the one or more processors, based on the set of coordinates, a 3′ overhang length for the first strand of the DNA duplex molecule, a 5′ overhang length for the first strand of the DNA duplex molecule, a 3′ overhang length for the second strand of the DNA duplex molecule, and/or a 5′ overhang length for the second strand of the DNA duplex molecule.
Embodiment 48. The method of any one of embodiments 45-47, comprising determining, using the one or more processors, a methylation status for one or more bases in the DNA duplex.
Embodiment 49. The method of any one of embodiments 45-48, comprising determining, using the one or more processors, a methylation status for one or more bases in a 3′ overhang or a 5′ overhang of the DNA duplex.
Embodiment 50. The method of any one of embodiments 45-49, wherein the DNA duple molecule is obtained from an individual, and the method further comprises comprising generating, using the one or more processors, a DNA duplex molecule overhang profile for the individual comprising information about overhangs for a plurality of DNA duplex molecules.
Embodiment 51. The method of embodiment 50, wherein the information comprises, for a plurality of DNA duplex molecules, the 3′ overhang length for the first strand of the DNA duplex molecule, a 5′ overhang length for the first strand of DNA duplex molecule, a 3′ overhang length for the second strand of the DNA duplex molecule, and/or a 5′ overhang length for the second strand of the DNA duplex molecule.
Embodiment 52. The method of embodiment 50 or 51, wherein the information comprises a sequence of a 5′ overhang or a 3′ overhang.
Embodiment 53. The method of any one of embodiments 49-52, wherein the information comprises (1) a ratio, or a distribution of ratios, of the a 5′ overhang length for the first strand of DNA duplex molecule to a 3′ overhang length for the first strand of DNA duplex molecule, (2) a ratio, or a distribution of ratios, of the a 5′ overhang length for the first strand of DNA duplex molecule to a 3′ overhang length for the second strand of DNA duplex molecule, (3) a ratio, or a distribution of ratios, of the a 5′ overhang length for the first strand of DNA duplex molecule to a 5′ overhang length for the second strand of DNA duplex molecule, (4) a ratio, or a distribution of ratios, of the a 3′ overhang length for the first strand of DNA duplex molecule to a 5′ overhang length for the second strand of DNA duplex molecule, or (5) a ratio, or a distribution of ratios, of the a 3′ overhang length for the first strand of DNA duplex molecule to a 3′ overhang length for the second strand of DNA duplex molecule.
Embodiment 54. The method of any one of embodiments 49-53, wherein the information comprises a ratio, or a distribution of ratios, of (1) a 5′ overhang length for the first strand of DNA duplex molecule, a 3′ overhang length for the first strand of DNA duplex molecule, a 5′ overhang length for the second strand of DNA duplex molecule, a 3′ overhang length for the second strand of DNA duplex molecule to (2) a duplex length.
Embodiment 55. The method of any one of embodiments 50-53, further comprising comparing, using the one or more processors, the DNA duplex molecule overhang profile to a reference DNA duplex molecule overhang profile.
Embodiment 56. The method of embodiment 55, wherein the reference DNA duplex molecule overhang profile is based on DNA duplex molecules from a normal sample or a plurality of normal samples.
Embodiment 57. The method of embodiment 55, wherein the reference DNA duplex molecule overhang profile is based on DNA duplex molecules from a sample obtained from an individual with cancer or a plurality of individuals with cancer.
Embodiment 58. The method of embodiment 55, wherein the reference DNA duplex molecule overhang profile is based on DNA duplex molecules from a sample obtained from an individual with an abnormal fetus or a plurality of individuals with an abnormal fetus.
Embodiment 59. The method of embodiment 55, wherein the reference DNA duplex molecule overhang profile is based on DNA duplex molecules from a sample obtained from an individual that received a stable transplant or a plurality of individuals that received a stable transplant.
Embodiment 60. The method of embodiment 55, wherein the reference DNA duplex molecule overhang profile is based on DNA duplex molecules from a match normal sample obtained from the individual.
Embodiment 61. The method of embodiment 55, wherein the reference DNA duplex molecule overhang profile is based on DNA duplex molecules from a prior sample obtained from the individual.
Embodiment 62. The method of any one of embodiments 45-61, wherein the DNA duplex molecule is a cell-free DNA molecule.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method of making a nucleic acid construct comprising:
directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule, to provide a circular nucleic acid construct; and
converting (i) non-methylated cytosine residues in the circular nucleic acid construct into uracil residues, or (ii) methylated cytosine residues in the circular nucleic acid construct into uracil residues or dihydrouracil residues, wherein a duplex structure of the DNA duplex molecule is transformed into a single-stranded structure.

2. The method of claim 1, wherein the DNA duplex molecule is a cell-free DNA duplex molecule.

3. The method of claim 1, comprising phosphorylating the 5′ end of the first strand of the DNA duplex molecule and the 5′ end of the second strand of DNA duplex molecule.

4. The method of claim 3, wherein the 5′ end of the first strand of the DNA duplex molecule and the 5′ end of the second strand of the DNA duplex molecule are phosphorylated using a T4 polynucleotide kinase.

5. The method of claim 1, wherein the 3′ end of the first strand of the DNA duplex molecule is directly attached to the 5′ end of the second strand of the DNA duplex molecule, and the 3′ end of the second strand of the DNA duplex molecule is directly attached to the 5′ end of the first strand of the DNA duplex molecule, using a single-stranded DNA (ssDNA) ligase.

6. The method of claim 1, comprising fraying the ends of the DNA duplex molecule while maintaining a partial duplex of the DNA duplex molecule prior to directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule.

7. The method of claim 1, wherein non-methylated cytosine residues in the circular nucleic acid construct are converted into uracil residues using a bisulfite reaction or an enzymatic reaction.

8. The method of claim 1, comprising amplifying the circular converted nucleic acid construct, thereby forming a linear converted nucleic acid construct, wherein the uracil residues or dihydrouracil residues in the circular converted nucleic acid construct are recognized as thymine residues in the amplifying.

9. The method of claim 8, wherein the linear converted nucleic acid construct is a rolling circle amplification (RCA) product, a double-stranded polymerase chain reaction (PCR) product, or a double-stranded inverse PCR product.

10. The method of claim 8, wherein the amplifying comprises using a first primer targeting a converted first sequence and a second primer targeting a converted second sequence.

11. A method comprising sequencing the linear converted nucleic acid construct made according to the method of claim 8 to provide sequencing data.

12. The method of claim 11, wherein the sequencing data comprises paired-end sequencing data comprising a first and a second read.

13. The method of claim 12, further comprising aligning a first portion of the first read and a first portion of the second read to a first converted nucleic acid reference sequence, and aligning a second portion of the first read and a second portion of the second read to second converted nucleic acid reference sequence, wherein the first converted nucleic acid reference sequence and the second converted nucleic acid reference sequence are complements to each other when compared in an unconverted state.

14. The method of claim 13, further comprising determining a set of genomic coordinates within the first and second converted nucleic acid reference sequences, comprising a first genomic coordinate for the first portion of the first read, a second genomic coordinate for the first portion of the second read, a third genomic coordinate for the second portion of the first read, and a fourth genomic coordinate for the second portion of the second read.

15. The method of claim 14, further comprising determining, based on the set of genomic coordinates, a presence or absence of a 3′ overhang for the first strand of the DNA duplex, a 5′ overhang for the first strand of the DNA duplex, a 3′ overhang for the second strand of the DNA duplex, or a 5′ overhang for the second strand of the DNA duplex.

16. The method of claim 14, further comprising determining, based on the set of coordinates, a 3′ overhang length for the first strand of the DNA duplex molecule, a 5′ overhang length for the first strand of the DNA duplex molecule, a 3′ overhang length for the second strand of the DNA duplex molecule, or a 5′ overhang length for the second strand of the DNA duplex molecule.

17. The method of claim 11, comprising determining a methylation status for one or more bases in the DNA duplex molecule.

18. The method of claim 17, comprising determining a methylation status for one or more bases in a 3′ overhang or a 5′ overhang of the DNA duplex molecule.

19. The method of claim 11, wherein the DNA duplex molecule is obtained from an individual, and the method further comprises comprising generating a DNA duplex molecule overhang profile for the individual comprising information about overhangs for a plurality of DNA duplex molecules.

20. A nucleic acid construct made according to the method of claim 1.

21. A method of generating a DNA duplex overhang profile, comprising:
generating a circular converted nucleic acid construct, comprising:
directly attaching a 3′ end of a first strand of a DNA duplex molecule to a 5′ end of a second strand of the DNA duplex molecule, and directly attaching a 3′ end of the second strand of the DNA duplex molecule to a 5′ end of the first strand of the DNA duplex molecule, to provide a circular nucleic acid construct; and
converting (i) non-methylated cytosine residues in the circular nucleic acid construct into uracil residues, or (ii) methylated cytosine residues in the circular nucleic acid construct int uracil residues or dihydrouracil residues, wherein a duplex structure of the DNA duplex molecule is transformed into a single-stranded structure;
amplifying the circular converted nucleic acid construct, thereby forming a linear converted nucleic acid construct, wherein the uracil residues or dihydrouracil residues in the circular converted nucleic acid construct are recognized as thymine residues in the amplifying;
sequencing the linear converted nucleic acid construct, wherein the sequencing provides sequencing data;
aligning, using one or more processors, to the sequencing data to a first converted nucleic acid reference sequence and a second converted nucleic acid reference sequence, wherein the first converted nucleic acid reference sequence and the second converted nucleic acid reference sequence are complements to each other when compared in an unconverted state;
determining, using the one or more processors, a set of genomic coordinates within the first and second converted nucleic acid reference sequences, comprising a genomic coordinates for a first portion, a second portion, a third portion, and a fourth portion of the sequencing data; and
detecting, using the one or more processors, based on the set of genomic coordinates, a presence or absence of a 3′ overhang for the first strand of the DNA duplex, a 5′ overhang for the first strand of the DNA duplex, a 3′ overhang for the second strand of the DNA duplex, and/or a 5′ overhang for the second strand of the DNA duplex.
